(19) United States
(12) Patent Application Publication
(10) Pub. No.: US 2022/0175339 A1
(43) Pub. Date: Jun. 9, 2022

(54) Acoustic Sensor System for Sensing Acoustic Information from Human and Animal Subjects

(71) Applicant: Loon Medical LLC, Northfield, MN (US)

(72) Inventors: Marie Ann Johnson, Northfield, MN (US); Gregory Stuart Johnson, Northfield, MN (US)

(21) Appl. No.: 17/545,633

(22) Filed: Dec. 8, 2021

Related U.S. Application Data
(60) Provisional application No. 63/205,402, filed on Dec. 8, 2020.

(51) Int. Cl. A61B 7/00; A61B 7/04; A61B 5/00

(52) U.S. Cl. CPC A61B 7/003; A61B 7/04; A61B 5/0077; A61B 5/684; A61B 2562/227; A61B 2503/40; A61B 2562/0204; A61B 2562/182; A61B 5/742

(57) ABSTRACT

The present invention provides acoustic sensor systems and methods for detecting acoustic information from a human or animal subject. An acoustic sensor system of the disclosure herein detects acoustic information and generates an analog electric signal corresponding to the information that is highly accurate.

### PRIORITY CLAIM

This application claims the benefit of U.S. Provisional Patent Application No. 63/205,402 filed on Dec. 8, 2020, titled “ACOUSTIC SENSOR SYSTEM FOR PULMONARY CONDITIONS AND CONGESTIVE HEART FAILURE,” the disclosure of which is hereby incorporated herein by reference in its entirety for all purposes.

### FIELD OF THE INVENTION

The present invention is in the field of acoustic sensor systems. More particularly, the present invention is in the field of acoustic sensor systems that include an acoustic sensor that detects acoustic information and generates an analog signal corresponding to the detected acoustic information, wherein the acoustic sensor system includes a coupling member that allows the acoustic sensor system to be connected to a computer device such as a smart phone, tablet, laptop computer, desktop computer, or the like.

### BACKGROUND

Humans and animals emit acoustic information, i.e., sounds, both when healthy and unhealthy. For example, sounds may be emitted by the heart as it pumps blood, by the pulmonary system (e.g., lungs and other parts of the respiratory system) during inhalation, exhalation, and even between inhalation and exhalation, by the abdomen, vocal cords, the blood circulatory system, the lymphatic system, and the like.

Acoustic information emitted by the body is helpful to assess the health of a person. Sounds associated with a particular body system may have differing characteristics depending on whether that body system is functioning properly or not. Further, particular conditions impacting health may be associated with particular kinds of abnormal body sounds, respectively. In other words, different health conditions may be associated with certain sound characteristics that are like a fingerprint to identify the condition. Consequently, body sounds are useful not only to detect that a person is ill, but body sounds can also be used to identify the condition causing the illness. For example, individuals with a respiratory condition may display an abnormal breathing pattern that can be detected by listening to acoustic information emitted by the pulmonary system at one or more locations on the human or animal subject. The type of abnormal breathing pattern can indicate the condition(s) at issue.

Health care providers often use a device such as a stethoscope to listen to pulmonary, heart, and other body sounds. Devices with acoustic sensors also may be used to detect acoustic information from a subject and then generate an electric signal corresponding to the detected sounds. For example, U.S. Pat. Nos. 8,827,920 and 9,968,329 describe acoustic sensing devices used in health care to detect sounds from a patient.

A variety of diseases or disorders may be responsible for abnormal breathing sounds. These include infectious diseases caused by viruses and bacteria such as influenza, pneumonia, tuberculosis, bronchitis, pleurisy, and bronchiolitis. Abnormal breathing may also be caused by conditions not necessarily brought about by pathogens, such as asthma, COPD, emphysema, cystic fibrosis, pulmonary edema, pneumoconiosis, interstitial lung disease, and mesothelioma.

It can be difficult for a non-medical professional to acquire high quality acoustic information from a human or animal patient wherein the detected acoustic information is of a quality sufficient to help evaluate, diagnose, treat, or otherwise care for a person who is or who might be ill. Despite being difficult, an accurate assessment of health conditions such as respiratory function is nonetheless important. Patient history accounts for 90% of treatment (or titration) decisions according to reports. For example, sounds made during breathing that are otherwise associated with particular lung disorders can be difficult or impossible to evaluate when the detected acoustic information is of poor quality.

The need to accurately detect acoustic information is even more paramount in cases where the patient is unable to communicate with the caregiver. For example, newborns, infants, toddlers, persons in a coma, and animals with abnormal breathing generally cannot communicate with a caregiver to describe their symptoms which would otherwise be useful in diagnosis. Therefore, in the case of a respiratory problem as one example, the symptoms of children of ages 0-7 often are generally represented by parent interpretation of the respiratory signs. Also, patients may have intermittent symptoms, like exercise-induced asthma, that may not be reproduced during an office visit with a health professional.

As such, health care providers often must evaluate a condition based on a description of past symptoms rather than upon directly observed symptoms. For example, based on patient or parent feedback and not on directly observed evidence, physicians often treat respiratory issues with bronchodilators. It is not surprising, therefore, that it has been reported that in 60% of cases the bronchodilators have no effect on the respiratory symptoms. Moreover, respiratory conditions are often triggered by weather, allergens, and/or air quality, which continually change so that the associated health conditions may not be exhibited during an office visit with a medical professional.

Even further, some respiratory conditions are brought about by viral infections, and such infections might be most effectively treatable with antiviral medications within a limited window following the onset of symptoms. Often, by the time the patient is able to visit a medical office and be diagnosed with a viral infection, the patent is outside the window where the antiviral would be most effective to promote recovery with reduced risks of the patient becoming even more ill.

The ability to accurately and timely detect acoustic information from a patient, such as sounds associated with an individual's respiratory condition, optionally along with documenting (e.g., capturing photo or video images of the subject) other symptoms of a condition at the time of detecting the acoustic information, and then to accurately transmit this information to a medical professional (such as by using a tele-medicine approach) or other caregiver for evaluation would be of great benefit to the early and/or effective diagnosis and treatment of a variety of health conditions.

### SUMMARY

The current disclosure provides inventive acoustic sensor systems and methods for detecting acoustic information from a human or animal subject. An acoustic sensor system of the disclosure herein detects acoustic information and generates an analog electric signal corresponding to the information that is highly accurate. This allows the detected information to be useful in the detection, evaluation, diagnosis, and/or treatment of a wide variety of health conditions in which health status impacts the character of the detected sounds. The acoustic sensor system is useful to detect sounds from a wide range of body sources, including the heart as it pumps blood, the pulmonary system (e.g., lungs and respiratory system) during inhalation, exhalation, and even between inhalation and exhalation, by the abdomen, vocal chords, the blood circulatory system, the lymphatic system, and the like. The present invention is particularly useful for detecting pulmonary-related sounds from the subject.

An acoustic sensor system of the disclosure herein can be configured to be removably or permanently attachable to a wide range of computing devices such as a smartphone, computer tablet, laptop computer, desktop computer, network terminal, other computer device incorporating a computer processor, or the like.

In one mode of practice, the acoustic sensor system is useful to acquire acoustic information, such as respiratory sounds, from a subject and covert these sounds to a corresponding analog electric signal which incorporates information comprising the frequency spectrum of the soundwaves generated by the subject in a suitable time period during one or more detection periods. The system generates an output comprising the analog signal and transmits the signal to at least one computing device. The computing device may incorporate program instructions that cause the information comprising the signal to be displayed, stored in a memory associated with the computing device, the sensor system, and/or a remote database, modified, transmitted to another computing device, or otherwise handled to assist in using the output analog signal and/or derivatives thereof to assist with evaluation, diagnosis, or treatment of the subject.

For example, the output analog signal can be stored in a smartphone or tablet memory optionally along with other information obtained from the subject (e.g., blood pressure, pulse, photo images, video images, oxygenation, blood glucose levels, and/or the like). Thereafter, the smartphone may be presented to a medical professional. Using an app (i.e., software program) on the smartphone, the medical professional may access and view the information on the smartphone. Alternatively, the data can be transferred to a computing device used by the medical professional.

As another example, the output analog signal optionally along with other patient information can be transferred to a computing device which then transfers the data to a remote computer device associated with a health care provider.

The acoustic sensor system and methods of the present invention are of great benefit for furthering a proper diagnosis of an individual with an illness, such as a respiratory condition, as the method of using the device to detect acoustic information can be carried out by a person who may not be a medical professional and at any location. For example, a child may be sick at home. The parent or caregiver can use the device to detect sounds from the patient. The data can be stored locally in a memory of a computing device linked to the device and/or transmitted to a remote computing device associated with a medical professional. The information can be provided to a medical professional for viewing in real time or for later analysis via storing the information in a memory of a computer device. Whether viewed in real time or later, the data is then useful for evaluation, diagnosis, and/or treatment of the subject.

The acoustic sensor system is easily configured to be readily used with a variety of smartphones, tablets, computers, and/or other computing devices. The system can also include an application program comprising program instructions to carry out steps that facilitate data acquisition and recording as well as other functions useful for diagnosis.

The acoustic sensor system includes components and a structure designed to facilitate fast, accurate collection of acoustic information from a subject. The device has a design that improves quality of the electric output, such as by minimizing interference otherwise caused by electromagnetic factors or data manipulation in conventional sensors that compromise the signal integrity. In turn, this more accurate data obtained from sounds of a subject improves evaluation, diagnosis, and treatment of health conditions.

In one embodiment the disclosure provides an acoustic sensor system that captures acoustic information from a subject. In preferred embodiments, the sensor system captures respiratory sounds. The acoustic sensor system includes a housing with first and second housing portions and an interior air space. In the acoustic device, an acoustic sensor is held in a first housing portion, is configured to detect acoustic information from the subject, and converts the detected acoustic information into an analog electric signal. The acoustic sensor includes a first sensor portion facing into the air space of the housing, and a second sensor portion facing outward from the housing. An electrical terminal interface is provided in the interior air space of the housing on the first sensor portion. The second sensor portion receives acoustic information from the subject. The sensor system includes an electrical coupling member positioned at the second housing portion of the housing that has (a) a first electrical terminal interface positioned inside the interior air space of the housing in a manner to provide an air gap in the air space between the electrical terminal interface of the acoustic sensor and the first electrical terminal interface of the electrical coupling member; and (b) a second electrical terminal interface outside the housing that is configured to couple the electrical coupling member to a computing device.

The first electrical terminal interface of the electrical coupling member and the electrical terminal interface of the acoustic sensor are electrically coupled in the air space such that the analog electric signal can be electrically transmitted from the acoustic sensor to the electrical coupling member so that the analog electric signal can be provided as an analog output from the second electrical terminal interface. When the electrical coupling member couples the sensor system to a computing device, the analog output may be transmitted to the computing device. The computing device also may use the connection to transmit electrical power, program commands, and the like to the sensor system.

In another aspect, the disclosure provides a method for detecting acoustic information from a subject. The method includes steps of a) using the acoustic sensor system of the disclosure to detect acoustic information from the subject at one or more locations; b) using the acoustic sensor system to convert the detected acoustic information into one or more corresponding analog signals; and c) using the acoustic sensor system to transmit the one or more analog signals to at least one computing device.

In another aspect the disclosure provides an acoustic sensor system that includes the acoustic sensor system with features as described herein, and a computer system configured to perform steps comprising displaying an image, said image comprising a representation of at least a portion of the human or animal subject and ii) one or more identified locations on the representation from which acoustic information is to be obtained from the subject, wherein, if the image includes a plurality of identified locations, the computer system is further configured to perform steps that cause the computer system to display information indicative of a sequence by which to detect acoustic information from the plurality of identified locations.

In another aspect the disclosure provides an acoustic sensor system that captures acoustic information from a human or animal subject, said sensor system the system including a) an acoustic sensor configured to detect acoustic information from the human or animal subject and convert the detected acoustic information into an analog electric signal, wherein the acoustic sensor comprises a flat frequency response in a range from 100 Hz to 2000 Hz; and b) an electrical coupling member, said electrical coupling member comprising a first electrical terminal interface electrically coupled to the acoustic sensor by at least one electrical wire and a second electrical terminal interface configured to couple the electrical coupling member to a computing device; wherein the electrical wire has a gauge of at least 18 gauge AWG or higher and wherein the electrical wire is coupled to the acoustic sensor by an electric attachment medium comprising Ag.

### BRIEF DESCRIPTION OF DRAWINGS

FIG. 1A is a cross sectional view of an acoustic device of the present invention.

FIG. 1B is a cross sectional view of an acoustic sensor portion of the device of FIG. 1A in greater detail.

FIG. 2A is a perspective view of a system of the present invention including an acoustic device and a smartphone.

FIG. 2B is a perspective view of a system of the present invention including an acoustic device, a smartphone, and an adaptor therebetween.

FIG. 3 is a cross sectional view of another embodiment of an acoustic device of the present invention.

FIG. 4 is a perspective view of the acoustic device of FIG. 1.

FIG. 5 is a flow chart of an embodiment of a method of acquiring and using respiratory information.

FIG. 6 is a flow chart of an embodiment of a method of acquiring and using respiratory information using a computer-based program.

FIG. 7 is a flow chart of an embodiment of a method of acquiring and using respiratory information using a smartphone application program.

FIGS. 8A and 8B are schematic illustrations of smartphones displaying information and images from an application program.

FIGS. 8C and 8D are schematic illustrations of a subject's torso and exemplary locations for placement of the acoustic sensor.

FIG. 9 is a table of respiratory sounds and domain and associated frequencies.

FIG. 10 is a table of lung sound characterization.

FIG. 11 is exemplary information based on acquisition of respiratory sounds from inhalation and exhalation events of a subject.

### DETAILED DESCRIPTION OF PRESENTLY PREFERRED EMBODIMENTS

The present invention will now be further described with reference to the following illustrative embodiments. The embodiments of the present invention described below are not intended to be exhaustive or to limit the invention to the precise forms disclosed in the following detailed description. Rather a purpose of the embodiments chosen and described is so that the appreciation and understanding by others skilled in the art of the principles and practices of the present invention can be facilitated.

A “subject” as described herein is a mammalian or non-mammalian organism. In preferred embodiments useful for pulmonary applications, the subject has lungs, and the subject may have their respiratory soundwaves acquired and converted to a corresponding analog electric signal using the device of the disclosure. In the disclosure the sensor system and method of using the sensor system are exemplified with reference to human subjects, but it can be understood from the disclosure that the sensor system and methods are equally applicable to any type of mammalian or non-mammalian organism having lungs.

The device of the disclosure is suitable for use on newborns, infants, toddlers, children, young adults, medium aged adults, and older adults. The term “subject” can be interchangeably used with the term “patient,” with the term “patient” being particularly applicable to those subjects that are under the treatment of a medical professional.

A “user” as described herein is an individual that carries out the acquisition of acoustic information such as respiratory soundwaves from a subject using the acoustic sensor system of the disclosure. The user can be the subject or an individual that is different than the subject, and can be either a medical professional or an individual that does not have any medical training. In some cases, the user can be the subject, if the subject is able to carry out use of the acoustic device and any associated component, such as a smart phone or computer by himself or herself

An aspect of the current disclosure relates an acoustic sensor system that is configured to detect respiratory sounds from a subject and convert the detected sounds to an analog electric signal. In many modes of practice, the analog electric signal is then transmitted to a computing device that may be a computer, tablet, or smart phone.

In an aspect, the acoustic sensor system (also “device” or “acoustic device” hereinafter) includes a housing with a first (often towards the subject or patient) housing portion that includes an acoustic sensor, a second housing portion (often towards a computing device) that includes a plug having a first end that is positioned in the second housing portion of the housing. The plug has a second end that is configured to be removably or permanently attached to a port in a computer, tablet, smart phone, or other computer device.

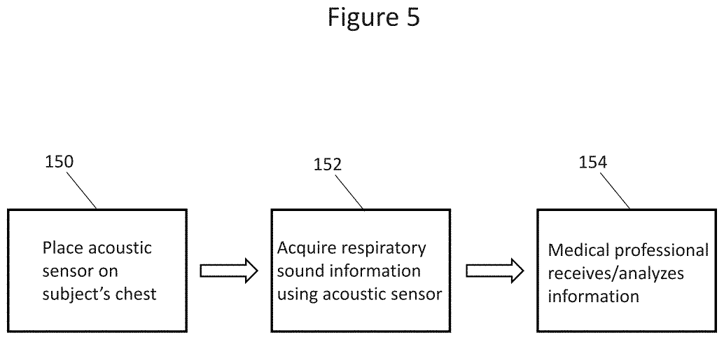

Another aspect of the current disclosure relates to method for detecting acoustic information, such as respiratory sounds, from a subject and optionally storing the acoustic information in a memory of at least one computing device. The method optionally can include one or more additional, various actions which facilitate collection of other kinds of information from the subject. The method can be performed by a person who may or may not be a medical professional to collect acoustic and other information from a subject who may have a health condition such as a respiratory condition, and then some or all of the information, or modifications thereof, can be presented to a medical professional for evaluation and diagnosis of a possible health condition, such as a respiratory condition, and then, if appropriate, medical treatment. In embodiments, basic steps in the method are set forth in FIG. 5, wherein an acoustic sensor is placed on a subject's chest (150), respiratory sound information is acquired using the acoustic sensor (152), and a medical professional receives and analyzes the information (154).

Another aspect of the current disclosure relates to a software application (such as an “app” used on a smartphone) that facilitates collection, display, modification, and/or other handling of information obtained from the subject. The app can receive, manipulate, utilize and record/store information received from the acoustic device of the disclosure. The app can also collect other information such as patent information about the subject, including information about the respiratory sound(s)and picture(s) and/or video(s) of the patient, blood pressure, pulse, oxygenation, blood glucose levels, or the like, and can also provide instructions or diagrams to facilitate the process of obtaining acoustic information from the subject/patient.

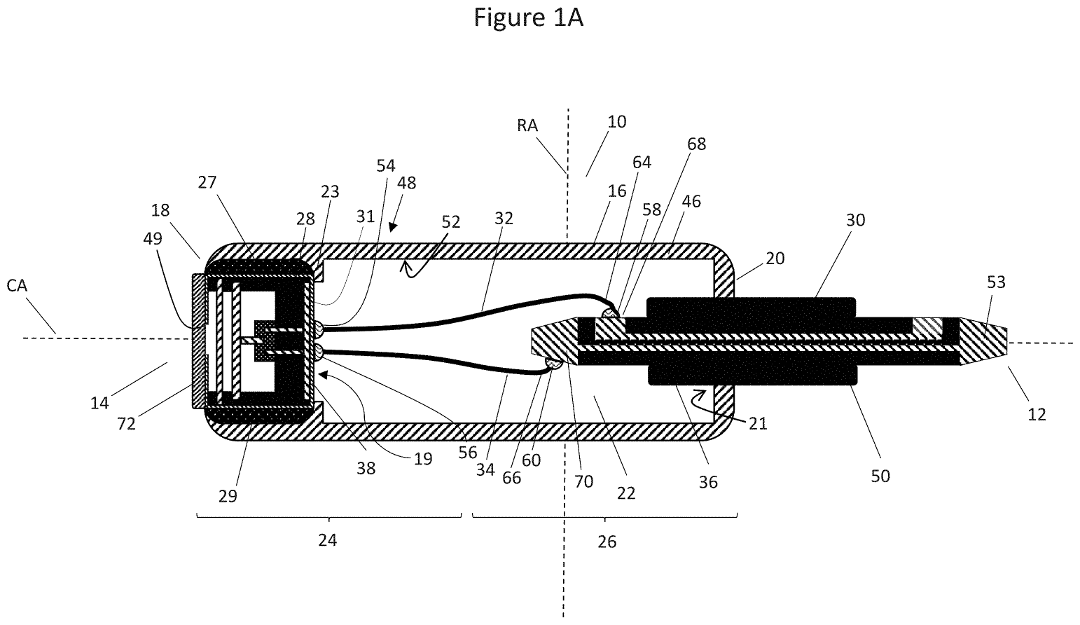

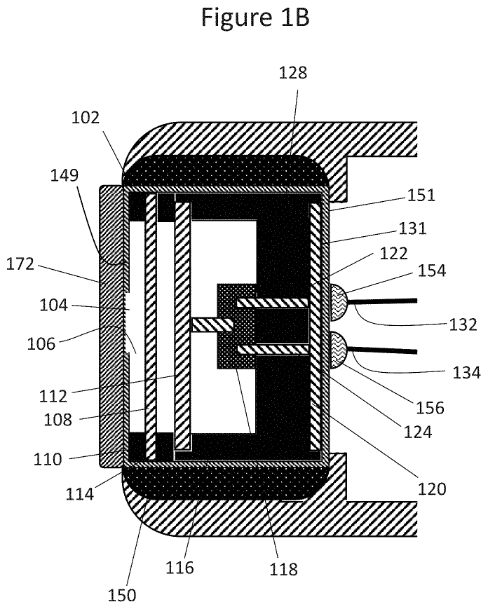

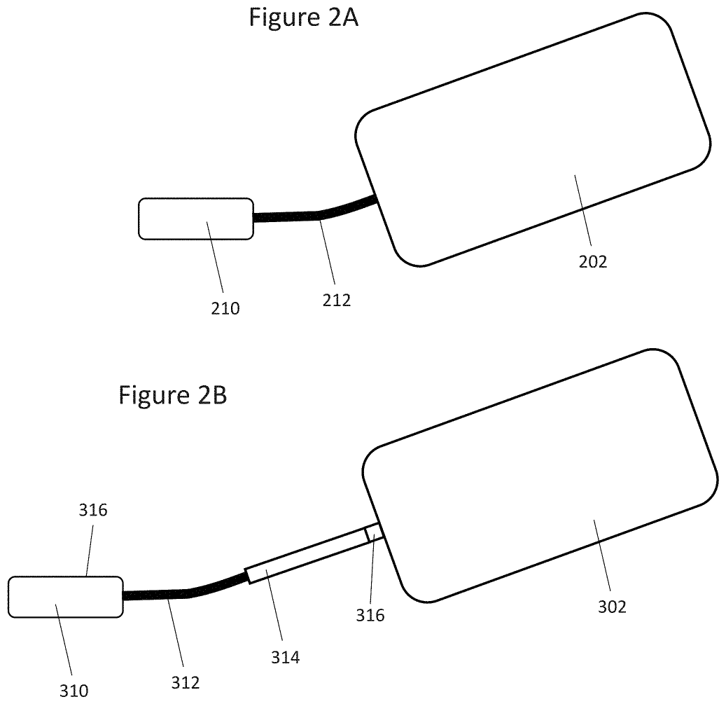

Reference to FIG. 1A is made schematically showing a cross-section of an embodiment of an illustrative acoustic device 10 of the present invention. The acoustic device 10 has a second (user) end 12 that is configured to be attached to a smartphone, tablet, computer, or other computing device (Not shown in FIG. 1A; rather, see FIG. 2A, showing computing device 102, acoustic device 110, and second end 112 that connects to computing device 102) and a first end 14 that is configured to detect respiratory sounds from the subject (subject/patient, not shown) and transmit a corresponding output electric signal to the connected smartphone or computer.

The acoustic device 10 includes a housing 16 that has a first end 18 (towards subject) and a second end 20 (towards user), and an interior cavity 22 within the housing 16. The housing 16 includes pocket 19 at a first end 18 of the housing 16 and an orifice 21 at second end 20 of the housing. The housing 16 can be described has having a first housing portion 24 which includes an acoustic sensor assembly 27 fit into pocket 19. The first housing portion 24 (also “sensor end” or “patient end”) often is oriented towards the patient or subject that is being monitored when in use. Optionally, the first housing portion 24 of the sensor system can include a feature to enhance collection of respiratory sounds from the subject, such as a cone (not shown) in accordance with conventional practices.

Towards the end of the first housing portion 24 is pocket 19 which holds the sensor assembly 27 in place. Pocket 19 can be formed by a lip 23 that is circumferential and projects radially inward from the inner surface 52 of the housing 16. Lip 19 generally projects inward a distance of not more than half, not more than a third, or not more than a quarter of the radius of the housing. Lip 19 extends partway into the interior cavity 22 but provides an opening so most or all of a second face 31 of the sensor assembly 27 faces inward towards the interior cavity 22. The lip 19 is sufficient to help hold the sensor assembly 27 in place at the end of the first housing portion 24 of the device 10.

Sensor assembly 27 includes acoustic sensor 28 fit into resilient gasket 29. Gasket 29 has an outer surface that is in contact with the inner surface 52 of the housing of the pocket 19, has an inner surface that is in contact with the outer surface of the sensor assembly 27, and holds sensor 28 circumferentially in place (also shown in greater detail in FIG. 1B). Sensor assembly 27 fits into pocket 19 so that a second face 31 of sensor 28 faces the cavity 22 and the first face 49 of sensor 28 faces outward from the pocket 19. Sensor 28 detects soundwaves coming from a subject and converts them to an electric signal, preferably an analog electric signal. Within the pocket 19, gasket 29 provides a resilient interface or buffer between housing 16 and sensor 28 and is particularly helpful when housing 16 is made of a harder or stiffer material than gasket 29. Consequently, gasket 29 desirably is less stiff and/or less hard than housing 16. It has been found that the analog electric signal developed by sensor 28 much more accurately represents the captured acoustic information when sensor 28 is indirectly in contact with housing 16 by the resilient interface provided by gasket 29. If sensor 28 is coupled to housing 16 directly, the accuracy of the developed analog electric signal with respect to the acoustic information is reduced.

The housing 16 can also be described has having a second housing portion 26 to which an electric coupling member in the form of plug 30 is mounted into orifice 21. In many modes of practice, the second housing portion 26 (also “second end” or “user end”) refers to a part of the housing 16 that is to be coupled to a smartphone, tablet, computer, or other computer device. For example, the second housing portion 26 of the acoustic device 10 will house a plug that is removably inserted into a port or jack of the smartphone, tablet, computer or other computing device. One purpose of plug 30 is to provide functionality so that device 10 can be connected to the port of a computing device (See FIG. 2).

Plug 30 includes a first portion 36 inside cavity 22 that is electrically coupled to sensor 28 so that plug 30 is able to receive the analog electric signal from the acoustic sensor 28 and which then relays the analog electric signal from the acoustic device 10 to a connected computing device 102 (see FIG. 2). Within the housing 10 the acoustic sensor 28 is electrically connected to the plug 30 by first wire 32 and second wire 34. Plug 30 is mounted to housing 16 so that a second plug portion 50 is outside the housing. The tip 53 of the plug at end 12 is configured to be plugged into a complementary port on a computing device. Optionally, tip 53 of the plug at end 12 can be plugged into an adaptor which carries the electric signal but changes the plug to a different plug type to be connected to a desired port on a computing device. For purposes of illustration, plug 30 is shown as an audio jack. Exemplary types of suitable audio jacks include Tip/Ring/Ring/Sleeve (TRRS), Tip/Sleeve (T/S), Tip/Ring/Sleeve (T/R/S), Tip/Ring/Ring/Ring/Sleeve (T/R/R/R/S) plugs, or the like.

An audio jack such as plug 30 is only one option for connecting device 10 to a computing device. Any other suitable connection form factor may be used such as USB, HDMI, lightning, VGA, DVI, display port, mini display port, parallel, serial, PS/2, firewire, thunderbolt, or the like.

In some embodiments, the plug 30 has a first portion that is held in place by the housing, a second portion comprising a connection that can be plugged into a port of a computer device, or an adapter, and an elongate flexible portion between the first and second portions (not shown). The elongate flexible portion can be in the form of a plastic-coated wire(s), and can be of any desired length. For example, the elongate flexible portion can be short (about a centimeter) or long (such as up to several meters), or any length in between. Longer elongate flexible portions can be particularly useful if the computer device to which the acoustic sensor is attached is stationary (e.g., a desk-top computer).

The housing 16 can be of any shape or configuration suitable to house the acoustic sensor 28 and at least a first portion of the plug 30 in a manner to have an air space of the cavity 22 between the second face 31 of the acoustic sensor 28 and the first portion 36 of the plug 30. In representative embodiments and as shown, the housing 16 has an elongate shape, meaning the length (along the central axis CA) of the acoustic device 10 from a first end 18 of the housing 18 to the second end 20 of the housing is greater than the width (along the radial axis RA) of the acoustic device. In embodiments, the length is 1.1-times or greater, 1.2-times or greater, 1.3-times or greater, 1.4-times or greater, 1.5-times or greater, 1.6-times or greater, 1.7-times or greater, 1.8-times or greater, 1.9-times or greater, or 2-times or greater than the width. In embodiments, the length is less than 20-times, less than 15-times, less than 10-times, less than 8-times, less than 7-times, less than 6-times, or less than 5-times than the width. The length to width ratio can be in the range of any of these values, e.g., a ratio in the range of 1.1:1 to 20:1, etc.

In exemplary embodiments the housing has a width (e.g., diameter) of at least 0.25 cm, at least 0.4 cm, such as in the range of about 0.25 cm to about 7.5 cm, about 0.4 cm to about 2.5 cm, or preferably about 0.55 cm to about 2.0 cm. In exemplary embodiments the housing has a length of at least 2.5 cm, at least 3.0 cm, such as in the range of about 2.5 cm to about 20 cm, about 3.0 cm to about 15 cm, or preferably about 3.75 cm to about 12.5 cm.

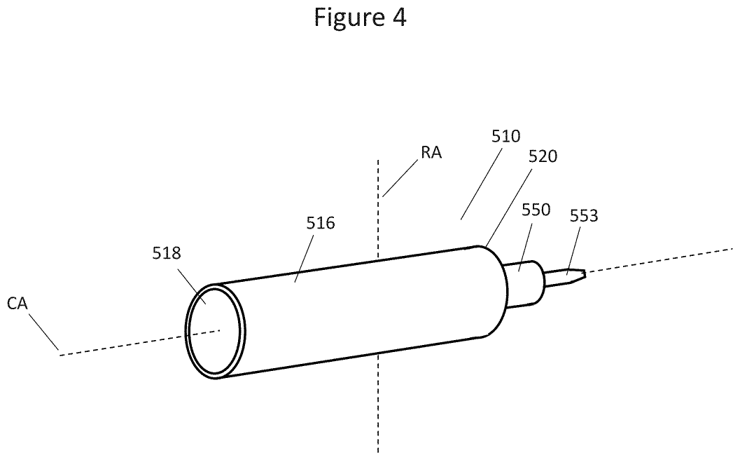

In many aspects, the housing 16 of the acoustic device 10 has a generally cylindrical shape with the tops of the cylindrical shape corresponding to the locations of the sensor 28 and the plug 30 and the cylindrical sidewall being the housing walls extending between the sensor 28 and plug 30. The cylindrical shape provides the sidewall of housing 16 with a curved outer surface. In such embodiments the acoustic sensor 28 also may have a cylindrical shape to easily fit within the cylindrical shape of the housing 16 and within the pocket 19. However, the cross section of the housing 16 can be curved but in a way that deviates from a circular such as an oval or oblong shape. Alternatively, the housing 16 cross section can provide a housing shape with one or more flat outer surfaces being contemplated. Viewed as a cross-section of the housing 16, the other shapes may be polygonal (hexagonal, octagonal, rectangular, square, etc.). In yet other aspects, the housing 16 may have one or more different cross-sectional shapes along its length. For example, the housing 16 may have a cylindrical shape towards its first end 18 and a shape that is partially non-cylindrical towards the second end 20. FIG. 4 shows a perspective view of an acoustic device 510 having a cylindrically-shaped housing 516, with first end 518, second end 520, second plug portion 550, and tip 553 of the plug. Central axis CA and radial axis RA are labelled.

Referring again to FIG. 1A, the housing 16 of the acoustic device 10 can provide rigidity and structure to hold the acoustic sensor 28 (which is held within the 19 pocket of the housing) and the plug 30 in desired, relative positions. In embodiments, the housing 16 is not easily bendable at a midsection of the housing (between the acoustic sensor 28 and the plug 30). The housing 16 can have sufficient rigidity to hold the sensor 28 and the plug 30 in a linear arrangement as shown and also to help maintain the interior cavity 22 (an air gap) between the sensor 28 and plug 30. For example, referring back to FIG. 1A, the acoustic device 10 has a central axis and the acoustic sensor and plug are aligned along a central axis CA of the device.

The housing 16 can be made from any suitable material, such as one or more metals, one or more metal alloys, one or more woven or non-woven fabrics, one or more polymers, one or more paper, one or more ceramics, glass, composites of these, and/or combinations thereof. Exemplary polymer materials may be thermoplastic or thermoset and may include, but are not limited to polyurethane, polyester, polyamide, polyimideamide, a fluorinated polymer, polyvinylchloride (PVC), polystyrene (PS), polypropyleneoxide (PPO), poly(meth)acrylic polymers, polyimide (PI), epoxy polymers, polyolefin, polyoxymethylene (POM), and glass-epoxy composite material. In embodiments, the housing may be semi-rigid or rigid. As used herein, the term polymer includes a compound including ten or more repeating units, wherein the repeating units may be the same or different. Some useful embodiments of polymers may have a weight average molecular weight in the range from 1000 to 1,000,000, preferably 2500 to 100,000.

In certain embodiments, if a polymeric material is used to make the housing of the acoustic sensor (or other parts of the acoustic device), it can optionally be described with regard to particular hardness value, which may be reported as a Shore A Value according to the testing standard ASTM D2240-02b. For example, the Shore A scale is a numerical range from 0 (a very soft polymeric composition) to 100 (a very hard polymeric composition). In some embodiments, the polymeric material of the housing 16 has a Shore A value of at least about 40, and preferably in the range of about 60 to about 100.

The housing 16 can have a wall thickness within from a wide range. If the wall is too thin, the housing 16 may not be strong enough to properly hold the sensor 28 and plug 30 in place while maintaining the air gap, and/or may not protect the sensor 28 and plug 30 adequately.

Above a certain thickness, marginal housing benefit may be realized, and the housing may become too heavy and/or too expensive. The actual thickness of the wall can depend on other factors including the type of material or materials used to make the housing, and the desired degree of rigidity of the housing. Balancing these concerns, a suitable wall thickness in many embodiments would be at least about 1 mm, and desirably in the range of about 1 mm to about 5 mm.

Still referring to FIG. 1A, the housing 16 has a cavity 22 of air space within the interior of housing 16. The sensor 28 and plug 30 are held by housing 16 to maintain an interior cavity 22 (an air gap) between the sensor 28 and the plug 30. It has been found that interior cavity 22 (air gap) promotes the quality of the analog electric signal developed by sensor 28 and transmitted to the plug 30. Without such an air gap 23 being maintained in the interior of housing 16, it is found that the ability of the analog electric signal to accurately represent the detected acoustic information is compromised. Through the airspace is first wire 32 and second wire 34 which electrically connect the acoustic sensor 28 to the plug 30 across the interior cavity 22 (an air gap) and inside housing 16.

As described herein, the housing 16 has a first portion 24 which includes the acoustic sensor 28 that detects soundwaves coming from a subject and converts them to an analog electric signal. As shown in FIG. 1A, the acoustic sensor 28 is positioned in the first portion of the housing 24 so sound waves enter the first face 49 of the sensor 28, and those sound waves are converted to an electric signal output that is transmitted to the plug 30. Sensor 28 includes a second face 31 that is inside housing 16 and faces inward into the cavity 22. The output signal travels from the sensor 28 via the electrical coupling in the form of wires 32 and 34. As a feature of the invention, the electrical coupling is inside the housing 16 in the cavity 22 and traverses across an air gap in the inner cavity 22 between sensor 28 and plug 30. When device 10 is connected to a computing device by inserting plug 30 into a complementary port on the computing device, the output signal can be transmitted to the computing device via plug 30.

The acoustic sensor 28 can be of any shape or configuration, but preferably a sensor configuration is provided to provide suitable detection and transmission of acoustic information, e.g., respiratory sounds, from a subject. In some embodiments, and as shown with reference to FIG. 1B, the acoustic sensor 128 has a cylindrical shape. The sensor 128 can have a curved first surface 150, and flat second face 51.

An electric coupling interface in the form of terminals 54 and 56 are incorporated onto second face 31 of the acoustic sensor 28. Wires 32 and 34 are coupled to terminals 54 and 56, respectively. An electric coupling interface in the form of terminals 58 and 60 are incorporated onto terminals 68 and 70 on the first portion 36 of plug 30 housed inside housing 16 in the cavity 22. Wires 32 and 34 are coupled to terminals 58 and 60, respectively.

A wide range of sensors may be used as acoustic sensor 28. In representative embodiments, acoustic sensor 28 is in the form of at least one microphone such as a dynamic microphone, a condenser microphone, and/or a ribbon microphone. A dynamic microphone converts sound energy into electric energy using a diaphragm linked to a coil of wire that moves through a magnetic field. The movement generates a voltage profile corresponding to the sound waves received by the diaphragm. A condenser microphone (also known as a capacitor or electrostatic microphone) typically includes a thin membrane in close proximity to a solid metal plate. The membrane and the plate form a capacitor The distance between the membrane and the plate changes in correspondence to sound waves that are received by the membrane. This generates an electric signal that corresponds to the sound. The structure often receives power from an outside source and is said to be phantom powered. A ribbon microphone uses a thin metal ribbon placed between magnets to convert sound energy into corresponding electric energy.

For pulmonary sound sensing, the microphone desirably has a flat frequency response at least in a range from 100 Hz to 2000 Hz. The frequency response of a microphone refers to the range of sound that a microphone can reproduce and how its output varies within a range. The frequency response may be represented by a response curve in accordance with industry practice in which the relative response in dB is plotted as a function of frequency. A frequency response chart according to one mode of practice is generated by testing the microphone in an anechoic chamber in front of a calibrated speaker. Pink noise is played. Pink noise refers to sound in which all frequencies in a tested range are emitted with equal energy. The microphone signal is routed to a spectrum analyzer and a frequency response chart is generated.

A flat response with respect to a range of frequencies indicates the microphone is substantially equally sensitive to all frequencies in a range of interest. A flat response in the frequency range of interest indicates that the microphone accurately reproduces the sound source with little or no variation from the original sound. In the practice of the present invention, a frequency response shall be deemed to be flat with respect to a frequency range when the relative response in dB in the range of interest is both 1) no more negative than −5 db, preferably no more negative than −3 db, and preferably no more negative than −2db, and even no more negative than −1 db and 2) no greater than 5 db, preferably no greater than 3 db, and preferably no greater than 2 db, and even preferably no greater than 1 db.

A microphone also can be less sensitive to certain frequencies. This occurs when the relative frequency response is more strongly negative than is associated with a flat response. In the practice of the present invention, a microphone is less sensitive to a frequency when the relative frequency response at that frequency is at least −5 db or more negative, preferably at least −10 db or more negative.

For pulmonary applications, acoustic sensors in the form of a microphone preferably have a flat relative frequency response in the range from 100 Hz to 2000 Hz. More preferably, a microphone with such a flat frequency response is less sensitive to frequencies below 100 Hz, preferably below 90 Hz, and more preferably below 80 Hz and/or is less sensitive to frequencies greater than 2000 Hz, preferably greater than 3000 Hz. By being less sensitive at such lower and/or higher frequencies, the microphone is more sensitive for pulmonary frequencies and less sensitive to other kinds of sound frequencies that might be emitted by a subject. As a result, the sound captured is a more accurate representation of the pulmonary sounds without being unduly altered, obscured, or otherwise affected by other sounds that might emanate from the body or ambient electrical noise.

Preferably, acoustic sensor 28 comprises a condenser microphone. Condenser microphones are compact and economical. Additionally, condenser microphones provide a signal that represents the captured sound with high accuracy. With an ability to be phantom powered, onboard power supplies such as a battery are not needed. This is an advantage, because onboard power can interfere with accurate conversion of sound energy into a corresponding electric signal. In a particularly preferred embodiment, the acoustic device uses an acoustic sensor 28 that comprises an electret condenser microphone. Electret condenser microphones are often very small and capsular (cylindrical) in shape can have a very wide frequency response (e.g., in the range of about 10 Hz to 30 kHz). Additionally, electret condenser microphones can be selected to have a flat frequency response in a range from 100 Hz to 2000 Hz, making them particularly suitable for pulmonary sound sensing. Accordingly, an electret condenser microphone can be circumferentially surrounded by gasket 29, which is then secured in pocket 19 in the first housing portion 24 of housing 16. For purposes of illustration, sensor 28 is in the form of an electret condenser microphone.

For purposes of illustration, sensor 28 is in the form of an electret condenser microphone. Reference to FIG. 1B is made showing details and electret condenser microphone embodiment of sensor 28 in the form of an electret condenser microphone 128. The electret condenser microphone 128 has an outer shell 102 (which may also be referred to as a capsule) and typically made of a hard material or materials, such as including a metal such as aluminum. On the first face 149 there is an opening 104 in the outer shell 102 which allows sound waves to enter the interior of the microphone, the opening 104 leading to an air space 106 that occupies a first part of the microphone 128. Proximal (i.e., towards the second end) to the airspace 106 is an electret disc 108 which is spaced from the second face 149 of the outer shell 102 using a metal washer 110 (i.e., the metal washer 110 is distal (i.e., towards the first end) to the electret disc 108). The electret disc 108 can be made of a metalized (e.g., gold) mylar film. Proximal to the electret disc 108 is a pick-up plate 112 of an amplifier module which is spaced from the electret disc 108 by a plastic spacer ring 114. Pick-up plate 112 is part of an amplifier module that includes an amplifier housing 116, typically made of plastic (to provide insulation for the pick-up plate 112), a transistor 118 that is in mechanical contact, with the pick-up plate 112, and a printed circuit board (PCB) 120 that is connected to the transistor 118 via first lead 122 and second lead 124.

The pick-up plate 112 has holes for displaced air to move through.

The electret disc 108 holds a fixed electric charge. The close spacing and surface areas of the of the electret disc 108 and pick-up plate 112 create a capacitor. Soundwaves in the air cause electret disc 108 to moves back and forth which in turn changes the distance to the amplifier module pick-up plate 112, thereby creating a voltage change. For generating a signal, with the transistor 118 the gate is connected to the pick-up plate 112, the source is connected to the ground, and the signal appears on the drain (a common source configuration). When the voltage at the gate varies this causes differences in conduction by the transistor, which changes current through the drain and producing a signal across the drain resistor.

On the second face 131 of the acoustic sensor the first lead 122 of the transistor 118 is the source (S) and the second lead 124 of the transistor 118 is the drain (D). In the acoustic device, and for connection with the plug, the source is in electrical connection with ground, and the drain is in in electrical connection with a resistor and power source.

Now referring back to FIG. 1A in some embodiments, the first end 14 of the acoustic device 10 has an impedance modulating interface through which the sensor detects the acoustic information. For example, the first end 14 of the acoustic device 10 can have a skin impedance cover 72 (shown as feature 172 in FIG. 1B). Biological tissues, such as skin, behave as conductors whose impedance varies with frequency. Skin impedance is the resistance of the skin to transmitting the electrical signal from the lung or heart to the sensing element of the electrode. It is generally understood that the acoustic impedance of a material is the product of the density and acoustic velocity of that material. Impedance matching maximizes power transfer and minimizes signal reflection from the signal source. An impedance mismatch may lead to an incoming wave being reflected back toward the source, thereby limiting the signal power. The acoustic impedance of water is close to that of the body. A membrane such as silicone encapsulating a material with properties similar to water provides such matching.

In some embodiments of the acoustic sensor system the impedance modulating interface provides the acoustic sensor with an impedance match with a surface of the human or animal subject such that the ratio of the impedance of the acoustic sensor with respect to detecting the acoustic information to the impedance of the subject surface with respect to transmitting the acoustic information is in the range from 1:1.2 to 1.2:1.

Optionally, the acoustic sensor can have one or more additional mechanical feature(s) on the first end 14 (toward subject) of the sensor that facilitate acquisition of respiratory sounds, and an interior cavity 22 within the housing 16. For example, the acoustic sensor 10 can have a diaphragm, cone, or head (not shown) attached to the first end 14, similar to a stethoscope cone.

Referring to both FIG. 1A and 1B, within the housing 10 the acoustic sensor 28 is electrically connected to the plug 30 by first wire 32 and second wire 34. As used herein, the term “wire” refers to an insulated conductor generally being a conductive metal line surrounded by a plastic casing. In particular, with reference to FIG. 1A, the second face 31 of the sensor has an electric coupling interface in the form of terminals 54 and 56 as attachment points to electrically connect the first wire 32 and second wire 34. Shown in greater detail in FIG. 1B, first lead 122 of the transistor 118 is coupled to first wire 132 (to provide the electric source) via terminal 154 (a solder point) and second lead 124 of the transistor 118 is coupled to second wire 134 (to provide the electric drain) via terminal 156 (another solder point).

In turn, referring back to FIG. 1A, the second end 64 of the first wire 32 is connected to terminal 68 at of the plug 30, and the second end 66 of the second wire 34 is connected to terminal 70 of the of the plug.

In embodiments, the end of the wires 32 and 34 are electrically coupled to the sensor 28 and the plug 30 using a suitable electrical attachment medium such as being soldered to the first and second terminals 54 and 56 of the acoustic sensor 28, and also to terminals 58 and 60 at the tip and sleeve of the plug 30. While any type of solder material can be used to make these connections, preferably the solder material includes silver. It has been found that solder comprising silver helps the sensor 28 have a flatter frequency response, particularly in a range from 100 Hz to 2000 Hz, as compared to using other types of solder. Hence, using a solder comprising Ag to electrically couple the sensor 28 and plug 30 proves a more accurate acoustic output signal, particularly in combination with an air gap of the interior cavity 22 between sensor 28 and plug 30.

Any type of wire or wires can be used to connect the acoustic sensor to the plug. It has been found that the wire gauge impacts the accuracy of the output electric signal developed by sensor 28. Thinner wires (higher gauge) tend to provide output signals that are more accurate representations of the input sound signal from the subject. In some embodiments, therefore, the wire or wires used to connect the sensor 28 and plug have a gauge of at least 18 American wire gauge (AWG) or higher. In some embodiments, the electrical wire has a gauge of at least 23 American wire gauge (AWG) or higher.

In a preferred embodiment, the acoustic device uses metal-shielded wire or wires 32 and/or 34 to connect the acoustic sensor to the plug. A “metal-shielded wire” refers to one or more plastic-coated wires further having a metal material surrounding the plastic-coated wire(s). The metal shielding may be in the form of a continuous metal layer (e.g., a metal tube or foil) around the plastic-coated wire, or a braided strands of metal, such as copper or aluminum, around one or more plastic-coated wire(s). In some instances, both foil and braided shielding are used. The metal shielding may also be surrounded with a further protective coating. The use of a shielded wire can improve signal quality and reduce interference, thereby providing more accurate data obtained from respiratory sounds of a subject. In other words, the metal shielding helps the sensor device 10 to produce an output electric signal with a flatter frequency response as compared to using wire without metal shielding.

Optionally, the acoustic device includes a ferrite bead feature surrounding all or a portion of an electrically conductive path of the sensor (not shown). The ferrite bead feature mate be a ferrite block, ferrite core, ferrite ring, EMI filter, or ferrite choke, partially or fully surrounding an electrical path, such as a wire leading from the acoustic sensor to the plug. Ferrite bead material is a type of choke that suppresses high-frequency electronic noise in electronic circuits, and use high-frequency current dissipation in a ferrite ceramic to for high-frequency noise suppression.

Referring back to FIG. 1A, in embodiments, the gasket 29 has a thickness (i.e., from its outer surface which contacts the inner surface of the housing 16, to its inner surface which is in contact with the outer surface of the acoustic sensor 28) effective to provide a resilient interface between sensor 28 and housing 16 in order to help acoustically isolate the sensor 28 from vibrations or other sound-affecting movement of housing 16. For example, in embodiments the thickness of the gasket is in the range of about 0.5 mm to about 10 mm, or more specifically in the range of about 1 mm to about 5 mm.

The gasket 29 can have one or more properties of being pliable, flexible, elastic, and/or resilient in order to help isolate sensor 28 from housing 16. The gasket 29 may be able to be deformed but return to an original shape without permanently altering the gasket's form. A resilient gasket can be made of a polymeric material that is resistant to permanent malformation, cracking, becoming brittle, and/or tearing. A rubber gasket 29 is a useful embodiment.

In certain embodiments, a polymeric material used to make the gasket can optionally be described with regard to particular Shore A Value according to the testing standard ASTM D2240-02b. The Shore A value of the gasket can be compared to the Shore A value of the housing, and in embodiments the Shore A value of the gasket is less than the Shore A value of the housing, meaning the gasket is made of a softer polymeric material. In some embodiments, the polymeric material of the gasket has a Shore A value of not greater than about 80, and preferably in the range of about 40 to about 70. In some embodiments the ratio of the Shore A hardness of the gasket 29 to the housing 16 is in the range from 0.6:1 to 0.95:1.

Desirably, the gasket 29 comprises at least one elastomeric polymer. Examples of different types of elastomeric materials that can be used to make the gasket portion of the device include, but are not limited to, styrene butadiene polymers (SBR), butyl rubber, silicone rubber (SiR), polyurethane (PU), saturated nitrile polymers (HNBR), nitrile butadiene polymers (NBR), fluoropolymers (e.g., Viton elastomer), chloroprene polymers (e.g., Neoprene), polyfluorosilicone (FSi), polyisoprene, and combinations thereof.

Referring back to FIG. 1A, in embodiments, the housing 16 has a second housing portion 26 which holds a first portion 36 of a plug in position so within the housing 22 so the tip 70 of the plug 30 can be pointed in the direction of the second face 31 of the acoustic sensor 28, facilitating electrical connection between the two. The alignment also helps to provide an output electric signal that more accurately represents the input sound signal.

Also shown in FIG. 1A, the plug 30 in the second portion 26 of the housing 16 includes a second plug portion 50 that extends outward from the end 20 of housing 16. The second plug portion 50 can be directly connected to an audio port of a computing device such as a smartphone or computer.

In some embodiments an adaptor may be used to provide a different kind of desired plug type for connection to a computing device such as a smart phone or computer. For example, the adaptor can receive second plug portion 50 and convert the connection to a different plug type for connection to a smartphone or computer. In some embodiments an adaptor is used to covert a TS, TRS, or TRRS plug to a plug such as an USB Type A plug, a USB Type C plug, a USB Micro plug, a USB Mini B plug, a USB Type Mini A plug, a Lightning plug, a Thunderbolt plug, a HDMI plug, a modem ethernet plug, or a display port plug. For example, an adaptor that changes a TRS plug to a Lightning plug or a USB Mini A plug can be referred to as TRS→Lightning or TRS→USB Mini A, respectively, herein. FIG. 2B shows a systems that includes the acoustic device 310 having a first plug type 312 (e.g., a TRS plug) proximal and external to the housing 316, an adaptor 314 that converts a first plug type to a second plug type 316 (e.g., TRS→USB Mini A), and a smartphone 302 which receives the second plug type 316.

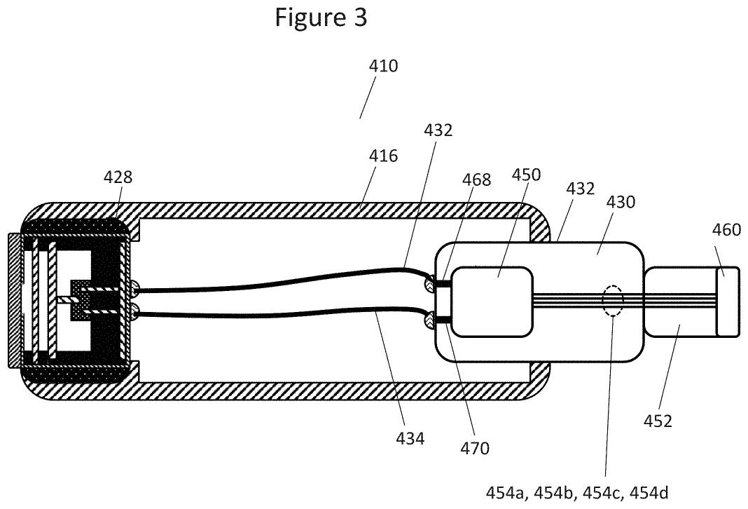

Instead of using an adapter, an alternative embodiment of a sensor system of the present invention includes a different type of plug for direct connection to a computing device. For example, an alternative device can incorporates a USB plug instead of plug 30, such as a TRS plug. In another embodiment, as shown in FIG. 3, the plug that is internal to the housing is a USB soundcard. In acoustic device 410 a USB soundcard 430 is placed in the proximal portion of the housing so the microphone port 468 and the auxiliary port 470 of the USB soundcard 430 are facing inwards towards the acoustic sensor 428 within the housing. The USB soundcard has a USB soundcard housing 432 that is held in place at the proximal portion of the device by the device housing 416. In the soundcard there are electrical connections from the microphone port 468 and the auxiliary port 470 to a soundcard circuit board 450, which may include an analog to digital converter (ADC), and an ISA or PCI interface. Wires 432 and 434 connect the microphone port 468 and the auxiliary port 470 to the acoustic sensor 428. Electrical connections (454a, 454b, 454c, 454d) lead from the circuit board 450 to pins of a USB plug 460 (e.g., a USB Type C plug).

Referring again to FIG. 1A, the acoustic device 10 may or may not include an onboard power source such as a battery. In the least, if a battery is included as a part of the acoustic device 10, in preferred aspects the acoustic device does not include a battery near the acoustic sensor 28. In more preferred aspects, the acoustic device 10 does not include any onboard power such as a battery. In certain embodiments, the absence of a battery near the acoustic sensor can improve device performance and the quality of the electric signal generated from the respiratory sounds. In certain embodiments, a battery such as in a smartphone or computer and located a distance from acoustic sensor provides a more desirable power source arrangement. In embodiments without onboard power, the sensor device 10 receives phantom power, if needed, from a computing device when the device 10 is connected to the computing device.

Acoustic sensor systems of the present invention, such as those shown in FIGS. 1A to 4, can be used in a variety of ways to help evaluate, monitor, diagnose, and/or treat a subject. Each device of the present invention may be used by one or more users with respect to one or more subjects. As noted herein a user of the acoustic device and any associated component (e.g. smartphone, computer, software) of a system that includes the acoustic device is one who operates the device and collects information from a subject and/or uses the collected data in some fashion such as to evaluate, monitor, diagnose, and/or treat the subject. A user may be onsite with the subject or at a remote location. A subject as used herein is any animal or human subject from whom acoustic information is obtained via the acoustic device.

In some embodiments, a subject may be an animal or patient in home, office, medical facility, or any other location that might have a health condition. The user(s) may include at least one onsite caregiver and/or at least one remote caregiver such as a medical professional. The onsite caregiver may help obtain the acoustic information from the subject and may be a person such as a parent, grandparent, sibling, other family member, nanny, work colleague, friend, medical professional making a house visit, or other person. In one example, the subject is a three-year-old child at home in a rural location. A parent at the home uses the device to collect respiratory sounds from the child. The data is provided to a remote medical professional in an urban region for remote monitoring, evaluation, diagnosis, and/or treatment. In another example, the is a three-year-old child at home in a rural location. A parent at the home uses the device to collect respiratory sounds from the child. The parent brings the child and the collected data to an appointment in the medical facilities of a medical professional.

While the user and subject can be different individuals, in other situations the user and the subject are the same individual. Based on the disclosure herein, it can be understood that a person may want to carry out a “self-test” wherein the individual uses the acoustic device to record her/his own respiratory sounds, optionally along with collecting other information useful for assessing the respiratory or other health condition of the individual.

The user of the device may or may not be a medical professional. Based on the disclosure herein, the acoustic device and any associated system components can be particularly useful for having a user who is not a medical professional record respiratory information from an individual, and then provide the information to a medical professional.

The same device may be used to collect and process acoustic information from more than one subject. The associated software may include a database to store information and data for each subject. Similarly, the same device may be used by more than one user from different class types. For example, one class type may be a family caregiver, while a second class type is a medical professional. The associated software may include functionality to provide a custom interface and functionality associated with each class of user. Due to the private nature of health information, a security profile may be used to control access to subject data so that only authorized persons can access the data for a particular subject.

The acoustic sensor is designed to be used with any computing hardware such as a computing device that includes an onboard computer processor or has the functionality to be used with a remote computer processor. The hardware can be, for example, a smartphone, tablet, remote computer terminal, laptop computer, or a desktop computer. Smartphones and tablet-type computers are particularly useful as they are able to be brought to the location of a subject and also easily maneuvered during the course of obtaining information from a subject. Moreover, such mobile devices can be used to collect pictures and or videos or other information of the subject before, during, and/or after the course of obtaining the acoustic information.

While commercially available smartphones and computers are preferred hardware, other hardware types can be used with the acoustic device, such as desktop computers and hardware having processors and user interfaces that are specifically designed for use with the acoustic sensor (e.g., hand held devices that are different than smartphones).

The computing device(s) can also include at least one associated software program specifically designed to be used with the acoustic device. On a smartphone the program can be in the form of an application, which can be downloaded on the smartphone prior to use of the acoustic device. The associated software may comprise program instructions to carry out steps related to one or more of controlling access to data and functionality, supplying power to the device, actuating the device on or off, adjusting settings on the device, calibrating the device, causing the device to detect sound information and to convert the sound into a corresponding electric signal, receiving data from the device, manipulating the data such as applying filters, converting analog data into digital data, storing data in a local memory on the phone and/or in a remote memory, incorporating the data into a database, displaying data or derivatives of the data, providing instructions for detecting sounds from the subject, guiding acquisition of other data about the subject, transmitting the data such as to a computing device associated with a medical professional, incorporating the data into a database, and the like.

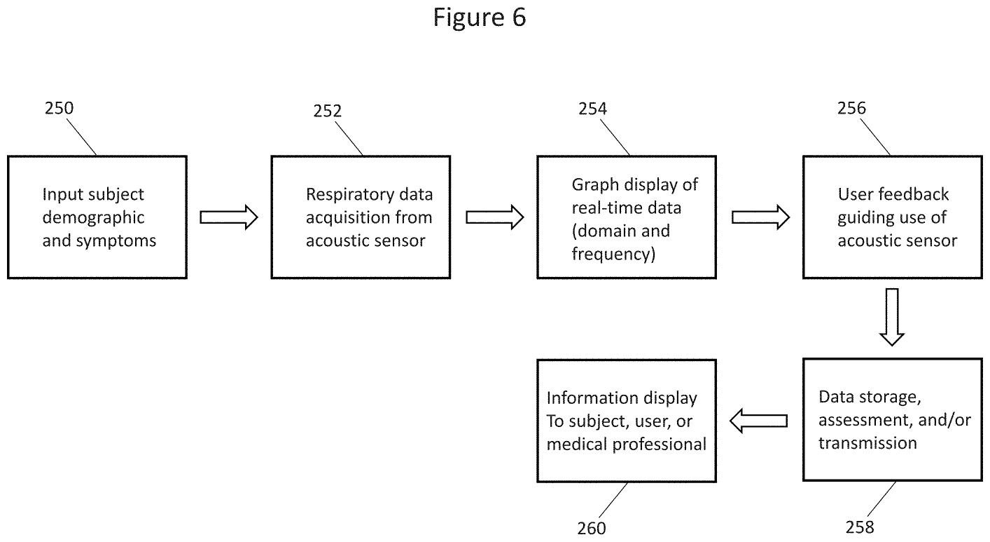

In embodiments, steps in the method implementing the acoustic sensor, computing device, and computer program are set forth in FIG. 6, wherein input regarding the subject's demographic information and symptoms are provided (250), respiratory sound information is acquired using the acoustic sensor (252), a graph display of real-time data is generated (254), feedback is provided guiding use of the acoustic sensor (256), data is stored, assessed, and/or transmitted (258), and information is displayed to the subject, user, or medical professional (260).

The software may include program instructions that carry out steps to provide user interface(s) associated with displays, keyboards, audio, touch, or the like. In some embodiments, the user interface is provided on a screen of the smartphone or computer. The displayed interface can include features that help the user to gather acoustic information from the subject. For example, the user interface may prompt the user to start and stop recording respiratory sounds from a subject at one or more locations on the subject. The interface on the display can also include features that instruct the user how and where to make respiratory recordings at one or more sites on the subject.

The interface on the display can also include features that prompt the user to enter other subject information, or non-subject information that may be relevant to process of recording respiratory sounds from the subject, such as date, time, temperature, relative humidity, local air quality, pulse, blood pressure, oxygenation, age, weight, height, gender, temperature, current or prior medications, medical history, recent travel, subject type (person, dog, cat, livestock, symptoms, photos or videos of the subject, etc.), and/or the like.

The application program can also present a diagram, image, or other representation of the subject, such as of the front or back of a torso, to provide guidance to the user for where the sensor should be placed on the subject. For example, prior to, during, after, or any combination thereof, pictures and/or videos of the patient can be taken, recorded, and associated with the subject's file, such as by using the smartphone app. A picture or video can be taken and associated with a particular segment of respiratory sound recording. The evaluation of an associated picture or video can help a medical professional make a more accurate evaluation, diagnosis, and/or treatment.

Additionally, photos, videos, or other images associated with a subject can help in the collection of sound information from the subject in the first instance. The application program can also present a diagram, image, or other representation of the subject, such as of the front or back of a torso, to provide guidance to the user for where the sensor should be placed on the subject. Program instructions in the interface may display the one or more photos in combination with graphic, video, written, audio, or other instructions for collecting the sound data. For example, the interface may display one or more photos or graphic representation of the subject and then visually show one or more locations on those subject representations for detecting sounds. If more than one location is to be used, the interface may include information, such as numbering, to indicate the order in which the sounds will be gathered at each location. For example, the diagram can include a plurality of locations on the torso of the subject directing the user to carry out respiratory sound analysis at a particular location and then to move the sensor to a new location on the torso and carry out the analysis. The steps of moving and analyzing can be carried out as needed and to complete analysis at all locations according to the diagram.

Subject information pertaining to the subject's physical state can also be obtained from one or more devices other than the acoustic device. For example, the subject's heart rate, blood pressure, and temperature can be measured by the subject and/or a user, and such information can be entered into the application program either manually or automatically (e.g., electronically, such as using Bluetooth or internet-of-things communications between electronic devices). Secondary medical analysis devices that can obtain such information include thermometers, heart rate sensors (including smartwatches and chest band sensors), sphygmomanometers, which are also commercially available as wireless devices which can transmit electronic information regarding one or more of heart rate, blood pressure, and temperature to the smartphone application.

The application program may also present questions to the user regarding the respiratory and other physical conditions. Such questions may include those relating to pain in any particular area of the body, shortness of breath, etc.

After plugging device 10 into the smart phone, device 10 may be automatically active in a manner similar to the way in which headphones are automatically active when plugged into a sound producing computing device. Alternatively, a power switch on device 10 (not shown) may be used to activate device 10. The caregiver uses the user interface to select a mode in which respiratory sounds are harvested from the subject.

To start analysis of respiratory sounds of a patient at a particular location, the first end of the device including the sensor is placed in contact or in close proximity, as appropriate, with the subject's skin. To ensure the smartphone or computer is receiving signal, the application program can be put in a test mode where there is no recording, but the signal generated from the respiratory sounds is still able to be visualized on the display screen or checked by the software in the background for appropriate quality. The subject can then inhale and/or exhale and the software can then guide or otherwise help the user to determine if the device and application are functioning properly and a desired signal is generated. Adjustments can be made, such as placement of the first end of the acoustic device, to obtain a useful signal. Once the device is ensured to be functioning properly, the program instructions to carry out steps for recording the soundwaves can commence.

At each location, sounds emitted by the subject are received by the acoustic sensor. The sensor converts the sounds into a corresponding analog electrical signal. Optionally, the device may be fitted with a component that helps to amplify the sounds received by the sensor. Desirably, no filtering is applied by the device to develop the analog electrical signal so that the signal accurately represents the sounds, although filtering may be used by the device or by the computing device connected to the device. The analog electric signal is transmitted to the electrical coupling member (such as plug 30 in FIG. 1A) via the electrical coupling between the sensor and the electrical coupling member. The air gap between the sensor and the electrical coupling member causes the two components to be spaced apart inside housing 16 so that electrical functioning of each component does not unduly interfere with the other component. For example, if the sensor 28 and plug 30 of FIG. 1A were too close, the interference could cause the frequency response of the sensor 28 to be less flat and, therefore, less accurate. The location of the air gap inside the housing 16 also further helps to isolate the components from interference to further help enhance the accuracy of the generated signal.

The device then transmits the analog electrical signal to the associated computing device such as a smartphone for further handling of the data. The associated software application includes program instructions that cause the data to be stored in a memory for later access. Desirably, the collected data is incorporated into a database associated with the subject.

The associated software includes program instructions to carry out steps that cause the results to be displayed on a computer device screen or other output device or format, and the associated software application can cause the results to be displayed in a desired format, such as one or more formats selected by a user. As an illustrative example, one format is a scatter graph, with points representing soundwaves of a particular frequency arranged along the y axis. Since a respiratory sound will include a plurality of soundwaves of different frequencies at any particular time, there will be a plurality of points spread out over a range on the y axis for a particular x value. The clustering of these points will allow the user to approximate an average frequency by visualization of the graph. For example, the graph can show time units (e.g., seconds) on the x axis, and then the frequency information (measured in Hertz, Hz, db, or the like) on the y axis. As another example, since respiratory sounds will produce a mixture of obtained frequencies, for any particular time point or time range, there will be frequency characteristics of the sounds as a function of a range of acoustic frequencies obtained from the subject. Hence, the interface may allow a collection of respiratory sounds from the subject to be displayed as a graph of respiratory sound frequency characteristics as a function of frequency, and/or the like. As another example, the computer application can determine the frequencies for a time point, and calculate the mean and/or median frequency, and optionally the frequency range, and display such data as a bar graph.

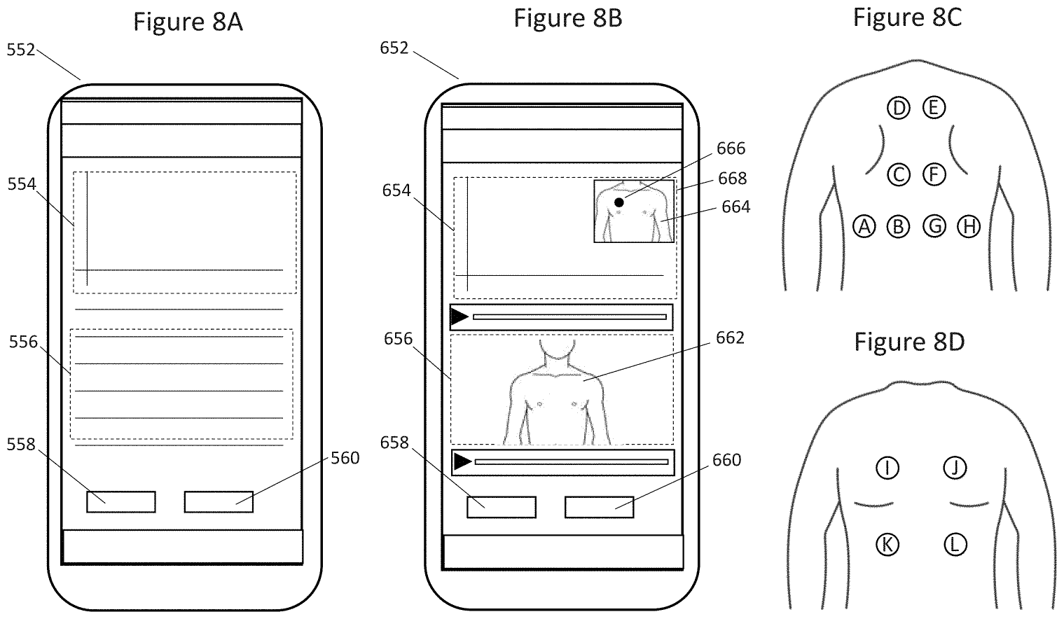

FIG. 8A is a schematic illustration of a smartphone 552 displaying certain functions and information generated from an application program and acquisition of respiratory information from a subject. This is one of many display modes the application program can provide. Display portion 554 generated by the application program shows a graph of sound frequencies over time. Display portion 556 shows sound intensity (measured in decibels) over time. Display portions 558 and 560 correspond to system activators which start and stop respiratory sound acquisition by the program.

FIG. 8B is a schematic illustration of another display mode the application program can provide on smartphone 652. Similar to FIG. 8A, display portion 654 generated by the application program shows a graph of sound frequencies over time, but also displays a schematic representation of the subject's torso 664 in window 668, and a location indicator 666 as to where the acoustic sensor should be placed on the patient's torso for acquiring respiratory information. Respiratory sound information acquisition is started and stopped using display portions 658 and 660 which corresponding to system activators as described with regards to FIG. 8A. Once acquisition at a particular torso location is accomplished, the location indicator shown on torso 664 moves, which instructs the user to move the acoustic device to the new location on the torso as indicated and acquire information. Display portion 656 generated by the application program allows a video of the actual subject 662 to be displayed and recorded. Video recording of the subject can be performed simultaneously with respiratory sounds acquisition at the torso location indicated in window 668, or video recording can be performed at a different time.

FIGS. 8C and 8D show exemplary locations on the back (A-H) and front (I-L) of the subject's torso that can be contacted with the acoustic sensor for respiratory sound acquisition. Torso locations (such as represented in FIGS. 8C and 8D) can be displayed in window 668 of the smart phone display as shown in FIG. 8B.

The computer program can also provide functionality for carrying out steps that store information indicative of frequencies of soundwaves from a subject with normal respiratory function. In some cases, a subject may record respiratory soundwaves during a period where no respiratory ailment is suspected, such as a time when the subject is in good health. At a later time when the subject is suspected of a respiratory ailment, a user can record the respiratory soundwaves from the subject and then compare them to the earlier soundwaves generated to determine if there is a change, which may be indicative of a respiratory ailment.

The system can be used to acquire respiratory information when the subject produces various types of abnormal respiratory sounds. In some cases the subject produces crackle noises during respiration. Crackles are related to air passing through pus and fluids, and related to alveolar sacs. Bibsilar cackles come from the base of the lungs and are a pneumonia indicator. Cackles are best detected during the first deep breaths at the lung bases posteriorly. Nath, A. R. and Capel, L. H. (1974). Thorax, 29, 223-227 describe inspiratory crackles early and late. Inspiratory crackles were recorded simultaneously with the inspiratory flow rate in patients with airways obstruction and in those with a restrictive defect. Early inspiratory crackles were associated with severe airways obstruction and late inspiratory crackles with a restrictive defect.

Respiratory noises such as rales and crackles (discontinuous sounds) and wheezes, rhonchi, and stridor (continuous sounds) can be produced during inhalation and/or exhalation.

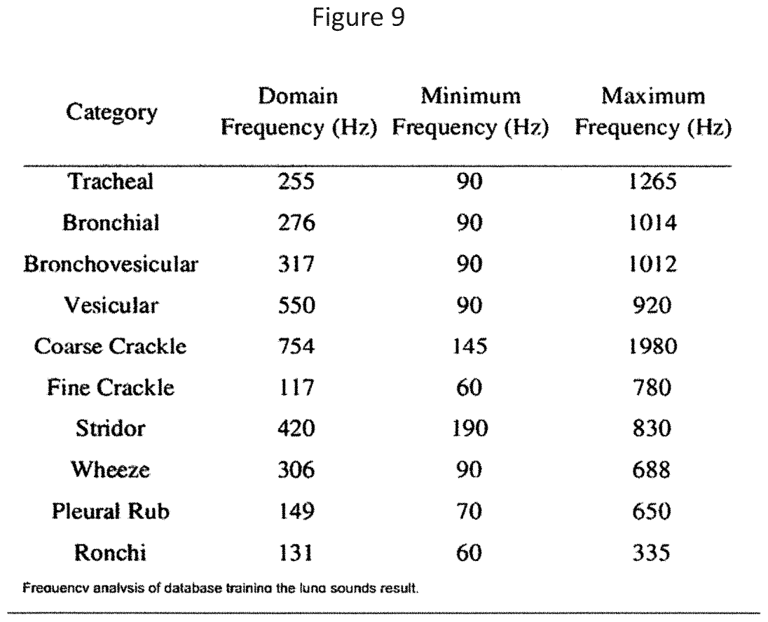

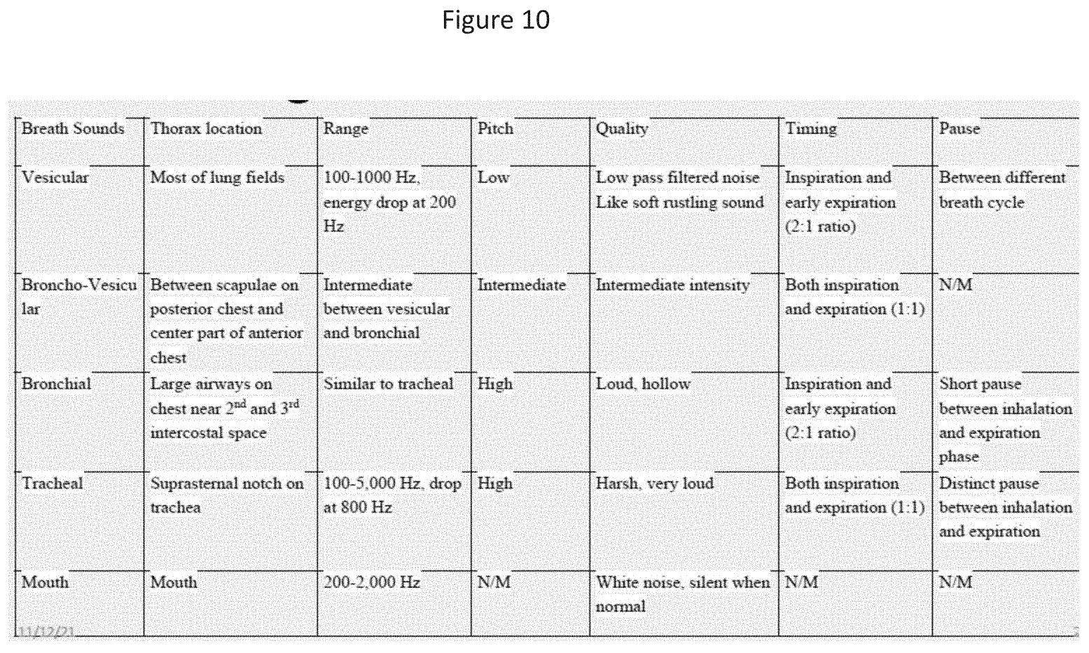

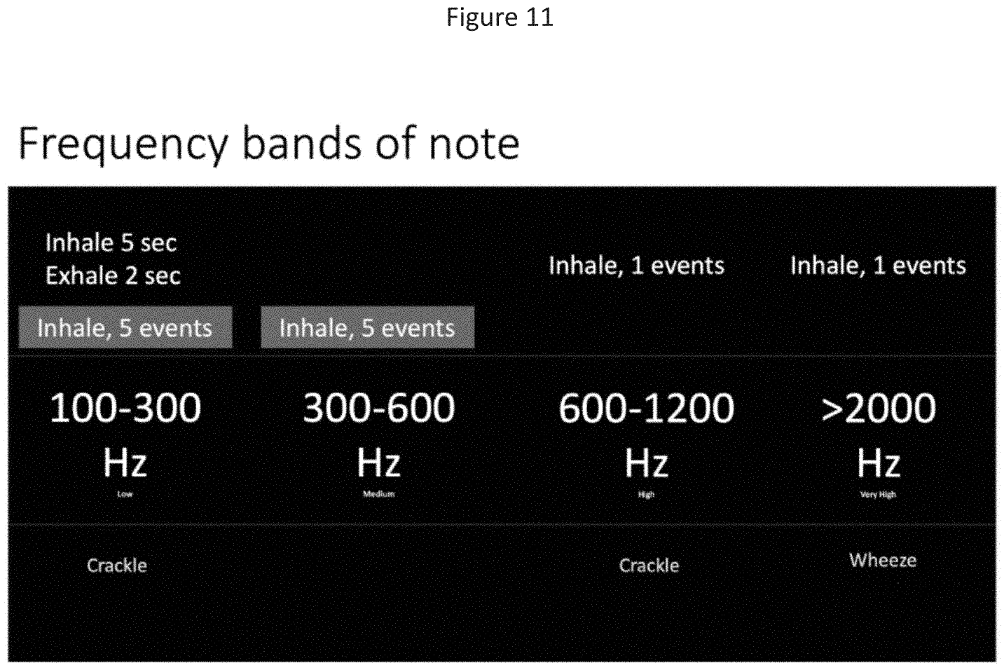

Rhonchi are continuous low-pitched, rattling lung sounds that can resemble snoring, and often heard in patients with CIPD, bronchiectasis, pneumonia, chronic bronchitis, or cystic fibrosis. Wheezes are caused by blockages to the main airways by mucous secretions, lesions, or foreign bodies. Rales are small clicking, bubbling or rattling sounds in the lungs. Stridor are wheeze-like sounds heard when breathing and often caused by a blockage of the windpipe (trachea) or in the back of the throat. These noises can provide soundwaves in certain frequency ranges such as 100-300 Hz (course crackle), 300-600 Hz (course crackle), 600-1200 Hz (fine crackle), and >2000 Hz (wheeze). FIG. 9 is a table showing categories of sounds and their domain, and minimum and maximum frequencies, from respiratory locations and conditions (from Pratama, D., et. al, 2020, Journal of Physics Conference Series 1500(1):012012). Lung sounds have unique signatures, and FIG. 10 is a table showing how lung sounds are characterized with sound location, frequency range, pitch, quality, timing, and pause. FIG. 11 is exemplary information based on acquisition of respiratory sounds from inhalation and exhalation events of a patient, and acoustic frequency ranges where abnormal respiratory sounds were detected. FIG. 11 is representative of results that can be obtained from methods of the disclosure.

In some modes of practice, the method does not necessarily require storage of the respiratory sounds of a subject. Rather, the software application includes program instructions effective to carry out steps generate graphed or otherwise displayed data in real time, and then this data is provided in real-time to a medical professional who is able to observe and understand the data. The medical professional can use the displayed data to evaluate, monitor, and/or diagnose the respiratory condition, and for potential treatment (real-time telemedicine). Based on the real-time information from the subject, the medical professional can ask the subject, or a user working with the subject, to perform further respiratory sound acquisition based on information received from the patient.

The medical professional can be in the same physical location as the subject, or can be in a different physical location. This latter mode of practice is particularly useful when the subject and medical professional are in different locations as the subject can be quickly diagnosed by the medical professional without requiring travel to and from a medical clinic.

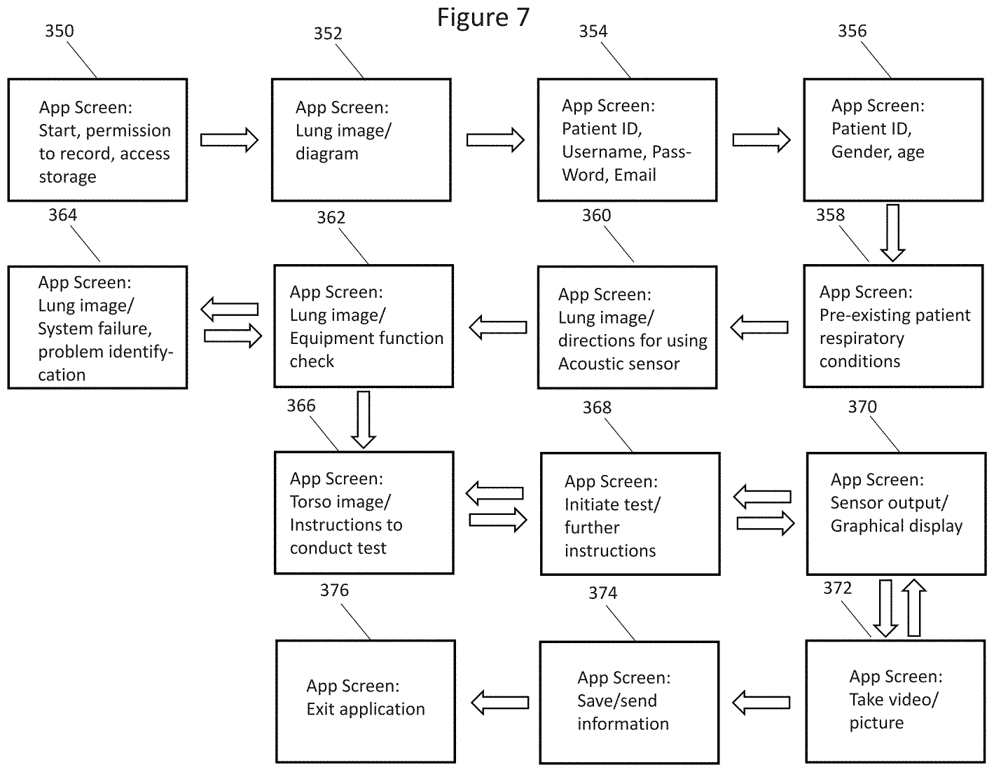

In some instances, the caregiver brings the smartphone and the data to a medical professional at a different location where the data can be evaluated. In some instances, the medical professional may be at the home and can evaluate the data at the home. In some instances, the app comprises program instructions that carry out steps causing the smartphone to transmit the data to a remote computing device associated with a medical professional. Using the data, the medical professional can evaluate, diagnose, and treat the patient. FIG. 7 provides steps of embodiments of the method of the disclosure using an application program as described herein.

All patents, patent applications, and publications cited herein are incorporated herein by reference in their respective entities for all purposes. The foregoing detailed description has been given for clarity of understanding only. No unnecessary limitations are to be understood therefrom. The invention is not limited to the exact details shown and described, for variations obvious to one skilled in the art will be included within the invention defined by the claims.

What is claimed is:

1. An acoustic sensor system that captures acoustic information from a human or animal subject, said sensor system comprising
a) a housing comprising second and first portions and an interior air space;
b) an acoustic sensor held at a first portion of the housing, said acoustic sensor configured to detect acoustic information from the human or animal subject and convert the detected acoustic information into an analog electric signal, and wherein the acoustic sensor comprises a first portion facing into the air space of the housing, and a second portion facing outward from the housing, wherein an electrical terminal interface is provided in the interior air space of the housing on the first portion and wherein the second portion receives acoustic information from the human or animal target; and
c) an electrical coupling member positioned at a second portion of the housing, said electrical coupling member comprising:
(a) a first electrical terminal interface positioned inside the interior air space of the housing in a manner to provide an air gap in the air space between the electrical terminal interface of the acoustic sensor and the first electrical terminal interface of the electrical coupling member; and
(b) a second electrical terminal interface outside the housing that is configured to couple the electrical coupling member to a computing device; and
wherein the first electrical terminal interface of the electrical coupling member and the electrical terminal interface of the acoustic sensor are electrically coupled in the air space such that the analog electric signal can be electrically transmitted from the acoustic sensor to the electrical coupling member so that the analog electric signal can be provided as an analog output from the second electrical terminal interface.

2. The acoustic sensor system of claim 1 wherein the acoustic sensor comprises a dynamic microphone.

3. The acoustic sensor system of claim 1, wherein the acoustic sensor comprises a phantom powered condenser microphone.

4. The acoustic sensor system of claim 1 wherein the acoustic sensor comprises a ribbon microphone.

5. The acoustic sensor system of claim 1, further comprising a resilient gasket that holds the acoustic sensor and wherein the gasket in turn is held by the housing in a manner effective to provide a resilient interface between the acoustic sensor and the housing.

6. The acoustic sensor system of claim 5, wherein the housing comprises a first material and the gasket comprises a second material, wherein the first material is harder than the second material.

7. The acoustic sensor system of claim 1, further comprising a computing device coupled to the second electrical terminal interface of the electrical coupling member.

8. The acoustic sensor system of claim 1 wherein at least an electrical wire electrically couples the acoustic sensor to the electrical coupling member, and wherein the electrical wire is connected to the acoustic sensor by an electrical attachment medium including silver.

9. The acoustic sensor system of claim 8 wherein the electrical wire comprises a metallic shielding.

10. The acoustic sensor system of claim 8, wherein the electrical wire comprises a gauge of at least 18 American wire gauge (AWG) or higher.

11. The acoustic sensor system of claim 8, wherein the electrical wire comprises a gauge of at least 23 American wire gauge (AWG) or higher.

12. The acoustic sensor system of claim 1, wherein the acoustic sensor comprises an electret condenser microphone.

13. The acoustic sensor system of claim 1 wherein the second portion comprises an impedance modulating interface through which the sensor detects the acoustic information.

14. The acoustic sensor system of claim 13, wherein the impedance modulating interface provides the acoustic sensor with an impedance match with a surface of the human or animal subject such that the ratio of the impedance of the acoustic sensor with respect to detecting the acoustic information to the impedance of the subject surface with respect to transmitting the acoustic information is in the range from 1:1.2 to 1.2:1.

15. The acoustic sensor system of claim 1, wherein the acoustic sensor comprises a flat frequency response in the range from 100 Hz to 2000 Hz.

16. The acoustic sensor system of claim 15, wherein the acoustic sensor is more sensitive with respect to frequencies in the range from 100 Hz to 2000 Hz than to frequencies below 100 Hz.

17. The acoustic sensor system of claim 15, wherein the acoustic sensor is more sensitive with respect to frequencies in the range from 100 Hz to 2000 Hz than to frequencies above 2000 Hz.

18. The acoustic sensor system of claim 1 wherein the analog output signal as transmitted from the electrical coupling member is unfiltered.

19. A method for detecting acoustic information from a subject, the method comprising
a) using the acoustic sensor system of claim 1 to detect acoustic information from the subject at one or more locations;
b) using the acoustic sensor system of claim 1 to convert the detected acoustic information into one or more corresponding analog signals; and
c) using the acoustic sensor system of claim 1 to transmit the one or more analog signals to at least one computing device.

20. The method of claim 19, wherein step a) comprises detecting pulmonary acoustic information from the human or animal subject.

21. The method of claim 19 wherein step a) comprises detecting pulmonary acoustic information from at least first and second locations of the human or animal subject.

22. The method of claim 19, wherein step a) comprises detecting pulmonary acoustic information from a range of from three to ten locations on the human or animal subject.

23. The method of claim 19 wherein step a) comprises detecting the acoustic information over a period of time such that the acoustic information comprises acoustic information for an inhalation and an exhalation.

24. The method of claim 19 further comprising the step of observing a representation of the subject that includes visual guidance of one or more locations on the human or animal subject from which to detect the acoustic information.

25. The method of claim 19 further comprising obtaining image information of the human or animal subject while detecting the acoustic information.

26. The method of claim 25, wherein the image information comprises video image information.

27. The method of claim 19, wherein step a) comprises using an image to help position the acoustic sensor system of claim 1 at the one or more locations, wherein the image comprises i) a representation of at least a portion of the subject and ii) one or more identified locations on the representation from which acoustic information is to be obtained from the subject.

28. An acoustic sensor system, comprising:
a) the acoustic sensor system of claim 1;
b) a computer system configured to perform steps comprising displaying an image, said image comprising a representation of at least a portion of the human or animal subject and ii) one or more identified locations on the representation from which acoustic information is to be obtained from the subject, wherein, if the image includes a plurality of identified locations, the computer system is further configured to perform steps that cause the computer system to display information indicative of a sequence by which to detect acoustic information from the plurality of identified locations.

29. An acoustic sensor system that captures acoustic information from a human or animal subject, said sensor system comprising
a) an acoustic sensor configured to detect acoustic information from the human or animal subject and convert the detected acoustic information into an analog electric signal, wherein the acoustic sensor comprises a flat frequency response in a range from 100 Hz to 2000 Hz; and
b) an electrical coupling member, said electrical coupling member comprising a first electrical terminal interface electrically coupled to the acoustic sensor by at least one electrical wire and a second electrical terminal interface configured to couple the electrical coupling member to a computing device; and
wherein the electrical wire has a gauge of at least 18 gauge AWG or higher and wherein the electrical wire is coupled to the acoustic sensor by an electric attachment medium comprising Ag.
